(19) United States
(12) Patent Application Publication
(10) Pub. No.: US 2013/0197414 A1
(43) Pub. Date: Aug. 1, 2013

(54) BRACE FOR CORRECTING CLUBFOOT

(71) Applicants: Michael Thomas Northen, San Francisco, CA (US); William Wheeler, San Francisco, CA (US); Emi Watanabe, Corte Madera, CA (US)

(72) Inventors: Michael Thomas Northen, San Francisco, CA (US); William Wheeler, San Francisco, CA (US); Emi Watanabe, Corte Madera, CA (US)

(21) Appl. No.: 13/481,250

(22) Filed: May 25, 2012

Related U.S. Application Data
(60) Provisional application No. 61/489,984, filed on May 25, 2011.

(51) Int. Cl. A61F 5/01

(52) U.S. Cl. CPC A61F 5/0127
USPC 602/29

(57) ABSTRACT

A brace for correcting a clubfoot condition includes a pair of foot restraints and a base member. Each of the pair of foot restraints includes a foot plate having a mounting feature on a bottom side of the foot plate and a number of apertures that traverse the mounting feature and the foot plate. Each of the pair of foot restraints also includes a first securing member with a first portion mechanically coupled to the foot plate, and an insert removably coupled to a top side of the foot plate. The base member includes a coupling feature positioned at each end of the base member, where each coupling feature includes a set of receiving apertures. The coupling features of the base member can be mechanically coupled to the pair of foot restraints using fastening devices that traverse the apertures in the foot plate, mounting feature, and the receiving apertures.

### CROSS-REFERENCE TO RELATED APPLICATIONS

This application claims priority under 35 U.S.C. §119 to U.S. Provisional Patent Application Ser. No. 61/489,984, titled “Brace for Correcting Clubfoot” and filed on May 25, 2011, the entire contents of which are hereby incorporated herein by reference.

### TECHNICAL FIELD

The present disclosure relates generally to orthopedic braces, and more particularly, to braces for correcting clubfoot.

### BACKGROUND

Clubfoot, also known as talipes equinovarus, is a congenital birth defect that causes one or both feet to turn inward. The exact causes of clubfoot are unknown, but research indicates genetic factors may play a role. Clubfoot results from abnormal development of the muscles, tendons, and bones of the fetus. Shortened tendons and ligaments on the inside of the lower leg lead to the foot turning inward. A tight Achilles tendon contributes to rigidity of the foot.

Clubfoot occurs in approximately 1 out of 750 births, with some variation across countries and ethnic groups and an increased incidence in children born to a parent with clubfoot. Clubfoot occurs more often in boys than girls. There are about 200,000 new cases of clubfoot each year around the world. For children born with clubfoot, both feet are affected in about 50% of the cases. Without treatment, children born with clubfoot cannot walk properly, if at all. Treatment can include the use of braces, casts, and/or surgery.

### SUMMARY

In general, in one aspect, the disclosure relates to a brace to correct a clubfoot condition. The brace can include a pair of foot restraints. Each of the foot restraints can include a foot plate having a mounting feature disposed on a bottom side of the foot plate and a number of apertures that traverse the mounting feature and the foot plate. Each foot restraint can also include a first securing member with a first portion mechanically coupled to the foot plate. Each foot restraint can further include an insert removably coupled to a top side of the foot plate. The brace can also include a base member mechanically coupled to each of the mounting features at each end of the base member. Each end of the base member can include a coupling feature, where each coupling feature includes at least two sets of receiving apertures.

In another aspect, the disclosure can generally relate to a method to correct a clubfoot condition using a brace. The method can include receiving a left foot into a left foot restraint. The method can also include wrapping a first securing member of the left foot restraint around a first portion of the left foot. The method can further include securing, after wrapping the left securing member around the first portion of the left foot, a first distal end of the left securing member to the left foot restraint. The method can also include receiving a right foot into a right foot restraint. The method can further include wrapping a second securing member of the right foot restraint around a second portion of the right foot. The method can also include securing, after wrapping the second securing member around the second portion of the right foot, a second distal end of the second securing member to the right foot restraint.

These and other aspects, objects, features, and embodiments will be apparent from the following description and the appended claims.

### BRIEF DESCRIPTION OF THE DRAWINGS

The drawings illustrate only exemplary embodiments of braces for correcting clubfoot and are therefore not to be considered limiting of its scope, as the braces for correcting clubfoot may admit to other equally effective embodiments. The elements and features shown in the drawings are not necessarily to scale, emphasis instead being placed upon clearly illustrating the principles of the exemplary embodiments. Additionally, certain dimensions or positionings may be exaggerated to help visually convey such principles. In the drawings, reference numerals designate like or corresponding, but not necessarily identical, elements.

FIGS. 1A and 1B show various views of an exemplary brace for correcting clubfoot in accordance with certain exemplary embodiments.

FIGS. 2A and 2B show various views of an exemplary base member for a brace used to correct clubfoot in accordance with certain exemplary embodiments.

FIGS. 3A-E show various views of an exemplary foot plate for a brace used to correct clubfoot in accordance with certain exemplary embodiments.

FIGS. 4A and 4B show various views of another exemplary foot plate for a brace used to correct clubfoot in accordance with certain exemplary embodiments.

FIG. 5 shows an exemplary foot restraint for a brace used to correct clubfoot in accordance with certain exemplary embodiments.

FIGS. 6-11B show various exemplary foot restraints for a brace used to correct clubfoot in accordance with certain exemplary embodiments.

FIGS. 12A and 12B show a portion of an exemplary brace for correcting clubfoot in accordance with certain exemplary embodiments.

FIG. 13 shows a flowchart of a method for correcting clubfoot in accordance with certain exemplary embodiments.

### DETAILED DESCRIPTION OF EXEMPLARY EMBODIMENTS

Exemplary embodiments of braces for correcting clubfoot will now be described in detail with reference to the accompanying figures. Like, but not necessarily the same or identical, elements in the various figures are denoted by like reference numerals for consistency. In the following detailed description of the exemplary embodiments, numerous specific details are set forth in order to provide a more thorough understanding of the disclosure herein. However, it will be apparent to one of ordinary skill in the art that the exemplary embodiments herein may be practiced without these specific details. In other instances, well-known features have not been described in detail to avoid unnecessarily complicating the description.

The braces for correcting clubfoot described herein may be used with feet of any size and/or shape. For example, exemplary embodiments of braces for correcting clubfoot can be used with newborn infants as well as teenager children. Further, an exemplary embodiment used to secure one foot to a foot restraint of a brace can be the same or a different exemplary embodiment used to secure the other foot to the other foot restraint of the brace.

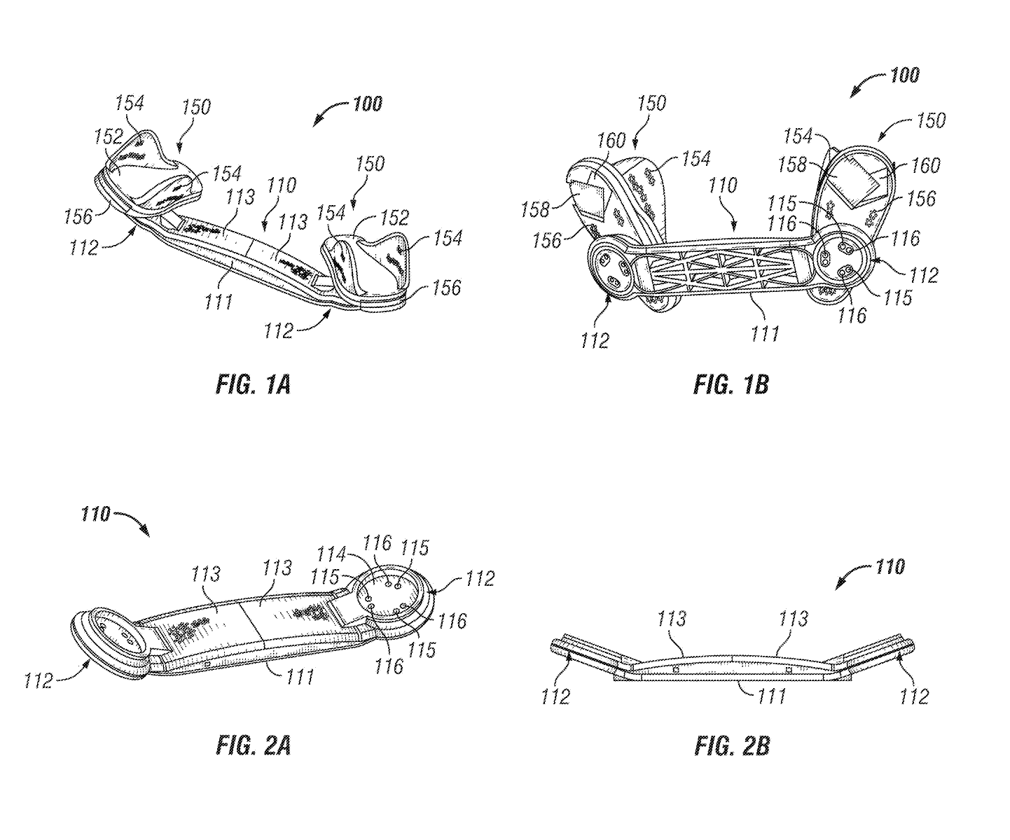

FIGS. 1A and 1B depict various views of a brace 100 for correcting clubfoot according to one exemplary embodiment. Specifically, FIG. 1A shows a top perspective view of the brace 100, and FIG. 1B shows a bottom perspective view of the brace 100. Referring now to FIGS. 1A and 1B, the brace 100 in this example includes a base member 110 and a pair of foot restraints 150 mechanically coupled to each end of the base member 110.

The base member 110 includes a set of coupling features 112 disposed on each end of the base member 110. Each coupling feature 112 can include a single set of receiving apertures or multiple sets of receiving apertures 115, 116. In FIG. 1B, there are two sets of receiving apertures 115, 116. Each set of receiving apertures 115, 116 can include at least two receiving apertures that are spaced equidistantly from each other in some geometric configuration (e.g., circle, square). In such a case, each foot restraint 150 can be mechanically coupled to the coupling feature 112 in a finite number of configurations. In this example, each set of receiving apertures 115, 116 has three receiving apertures that are spaced equidistantly in a circle.

Each receiving aperture traverses at least a portion of the coupling feature 112. In this example, each receiving aperture traverses the entire coupling feature 112. In certain exemplary embodiments, the coupling features 112 are set at an angle to (i.e., are non-linear with) the central portion of the base member 110. In certain exemplary embodiments, the angle between the coupling feature 112 and the central portion of the base member 110 can be adjusted. For example, the coupling feature 112 can be hingedly coupled to the central portion of the base member 110. In such a case, the hinged coupling can define a range of angles that may be discretely adjustable or freely adjustable. In addition, or in the alternative, the tilt (i.e., the offset angle between the plane of the base member 110 and the plane of the coupling feature 112) of the coupling feature 112 may also be adjusted.

In certain exemplary embodiments, each set of receiving apertures 115, 116 of the coupling feature 112 creates an outward angle when the foot restraint 150 is mechanically coupled to the coupling feature 112 of the base member 110. The outward angle can be measured relative to a perpendicular angle. The perpendicular angle may also be referred to as a normal angle and is the angle perpendicular to the axis along the length of the base member 110. The outward angles can be discrete angles that are predetermined based on the orientation of the sets of receiving apertures 115, 116. For example, if there are two sets of receiving apertures 115, 116, one outward angle corresponding to one set of receiving apertures 116 can be 30°, and another outward angle corresponding to another set of receiving apertures 115 can be 60°. There may also be other outward angles (e.g., 40°, 70°), either fixed or adjustable, that can exist.

The central portion of the base member 110 can include a bottom part 111 and a top part 113. The top part and the bottom part can be joined at approximately where a coupling feature 112 is disposed at each end of the base member 110. From this point, the bottom part 111 and the top part 113 can diverge, forming a dorsiflexion angle. In certain exemplary embodiments, the dorsiflexion angle can be adjusted. The bottom part 111 can be a solid piece and/or have a structural pattern. For example, as shown in FIG. 1B, the bottom part 111 of the base member 110 has a ribbed structural support where the ribs are criss-crossed. As another example, the bottom part 111 of the base member 110 can have a honeycomb shape.

Similarly, the top part 113 of the base member 110 can be a solid piece and/or have a structural pattern. The top part 113, bottom part 111, and coupling feature 112 can each be made of one or more of a number of materials, including but not limited to plastic, rubber, metal, and fiberglass. The top part 113, bottom part 111, and coupling feature 112 can be made from a single molded piece or be separate pieces mechanically coupled together. In certain exemplary embodiments, the top part 113 and/or the bottom part 111 can be adjustable along the length of the central portion. For example, the top part 113 and the bottom part 111 can each be slidably extendable and/or retractable to change the length of the base member 110 and/or the dorsiflexion angle.

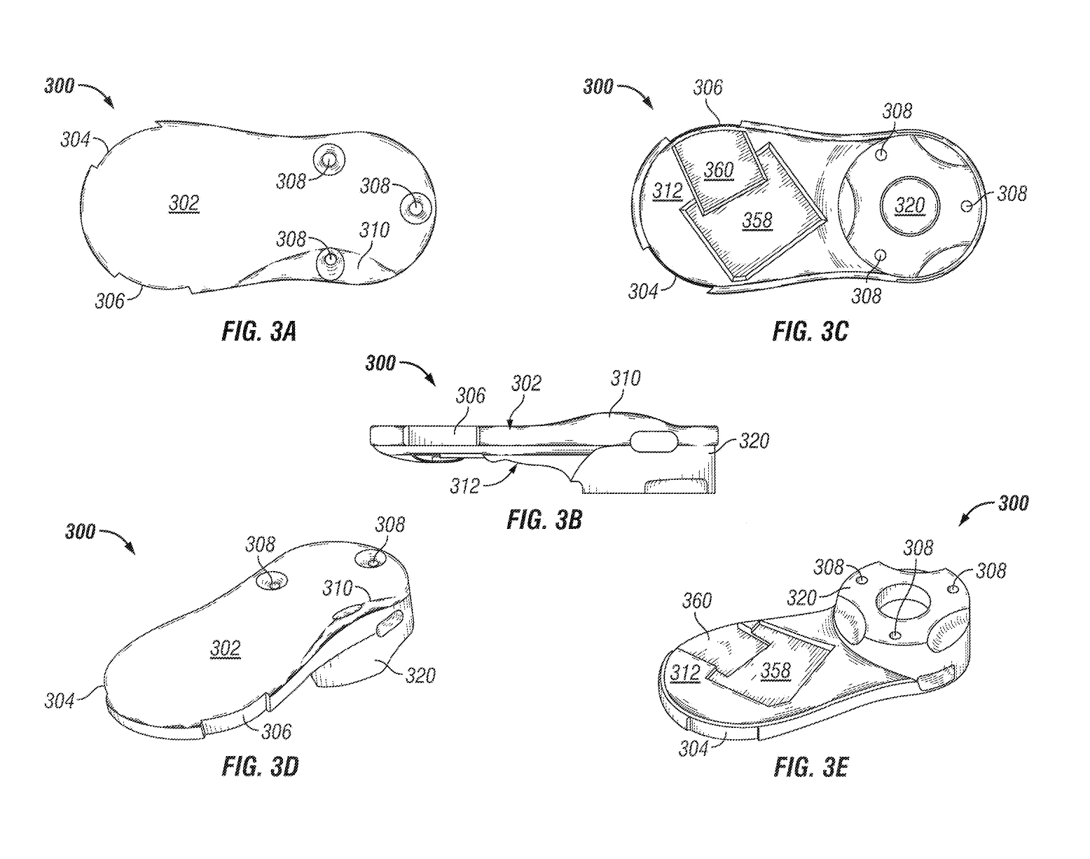

In certain exemplary embodiments, each of the pair of foot restraints 150 is complementary to each other and receives a foot that has a clubfoot condition. Each foot restraint 150 can include one or more of a number of features. For example, as shown in FIGS. 1A and 1B, the foot restraint can include a foot plate 156, an insert 152, one or more side supports 154. The foot plate 156 can include one or more of a number of features. For example, as shown in FIG. 1B, the bottom side of the foot plate 156 can include a strap guide 160 and a buckle inset 158. The foot plate 156 is described in further detail below with respect to FIGS. 3A-E.

The insert 152 of the foot restraint 150 is removably coupled to the top side of the foot plate 156. The insert 152 can be removably coupled to the top side of the foot plate 156 using none or one or more of a number of methods, including but not limited to snaps, Velcro, slotted fittings, and epoxy. The insert 152 can be made of one or more materials (e.g., fabric, gel, rubber, foam, metal) having any degree of flexibility and/or hardness. The shape of the insert 152 can be substantially the same as the shape of the top side of the foot plate 156.

Each side support 154 can be mechanically coupled to the foot plate 156 or the insert 154. The side support 154 can be removably or fixedly coupled to the foot plate 156 or the insert 154 using one or more of a number of methods, including but not limited to snaps, Velcro, slotted fittings, stitching, and epoxy. The side support 154 can be made of one or more materials (e.g., fabric, gel, rubber, foam, metal) having any degree of flexibility and/or hardness. The shape and/or dimensions (e.g., height, length) of the side support 154 can vary, depending on one or more of a number of factors, including but not limited to the severity of the clubfoot condition and the size of the foot.

FIGS. 2A and 2B show various views of an exemplary base member 110 for a brace used to correct clubfoot in accordance with certain exemplary embodiments. Specifically, FIG. 2A shows a top perspective view of the base member 110 described above with respect to FIGS. 1A and 1B, and FIG. 2B shows a side view of the base member 110 described above with respect to FIGS. 1A and 1B. Referring now to FIGS. 1 and 2, the top sides of the coupling features 112 of the base member 110 are shaped substantially the same as the mounting features of the foot plate, described in more detail below with respect to FIGS. 3A-E. In this example, the top side of the coupling features 112 are substantially circular. The two sets of receiving apertures 115, 116 are shown in FIG. 2A.

FIG. 2B shows the various non-linear angles formed by the various components of the base member 110. For example, the dorsiflexion angle formed between the top part 113 and the bottom part 111 of the base member is approximately 10°. Further, the angle formed between each coupling feature 112 and the bottom part 111 of the base member 110 is approximately 160°.

FIGS. 3A-E show various views of an exemplary foot plate 300 for a brace used to correct clubfoot in accordance with certain exemplary embodiments. Specifically, FIGS. 3A-C show a top, side, and bottom view of the foot plate 300. FIG. 3D shows a top perspective view of the foot plate 300, and FIG. 3E shows a bottom perspective view of the foot plate 300. Now referring to FIGS. 1-3, the foot plate 300 has a number (in this case, three) of apertures 318 that traverse the foot plate 300 as well as the mounting feature 320 disposed on the bottom side 312 of the foot plate 300.

In certain exemplary embodiments, the apertures in the foot plate 300 are arranged in a manner that corresponds to the configuration of a set of receiving apertures 115, 116 in a corresponding mounting feature 112 of the base member 110. The mounting feature 320 of the foot plate 300 can generally be shaped in a manner consistent with the shape of the corresponding mounting feature 112 of the base member 110. In this example, the mounting feature 320 of the foot plate 300 is circularly shaped to fit within the circular shape of the mounting feature 112 of the base member 110.

As alluded to previously, the foot plate 300 includes a top side 302 and a bottom side 312. Each of the top side 302 and a bottom side 312 can include one or more of a number of features. For example, as shown in FIG. 3A with respect to the top side 302, in addition to the apertures 318, an arch support feature 310 may be disposed on the top side 302. The arch support feature 310 can be made from a single molded piece or can be a separate piece mechanically coupled to the top side 302 of the foot plate 300. The arch support feature 310 can be of any shape and have any dimensions suitable to provide arch support to the foot received into the corresponding foot restraint 150.

The features disposed on the bottom side 312 of the foot plate 300 can include, in addition to the mounting feature 320, a channel 360 and a slot 358. In certain exemplary embodiments, the channel 360 and the slot 358 are located adjacent to each other on the bottom side 312. The channel 360 can be used to guide a securing member (described below with respect to FIGS. 5-12). As shown in FIGS. 3B-D, complementary to the channel 360 is a notch 306 disposed on the side of the foot plate 300 adjacent to the channel 360. The notch 306 is disposed along the path of the channel 360 along the outer edge of the foot plate 300. In certain exemplary embodiments, the notch 306 complements the channel 360 in guiding the securing member.

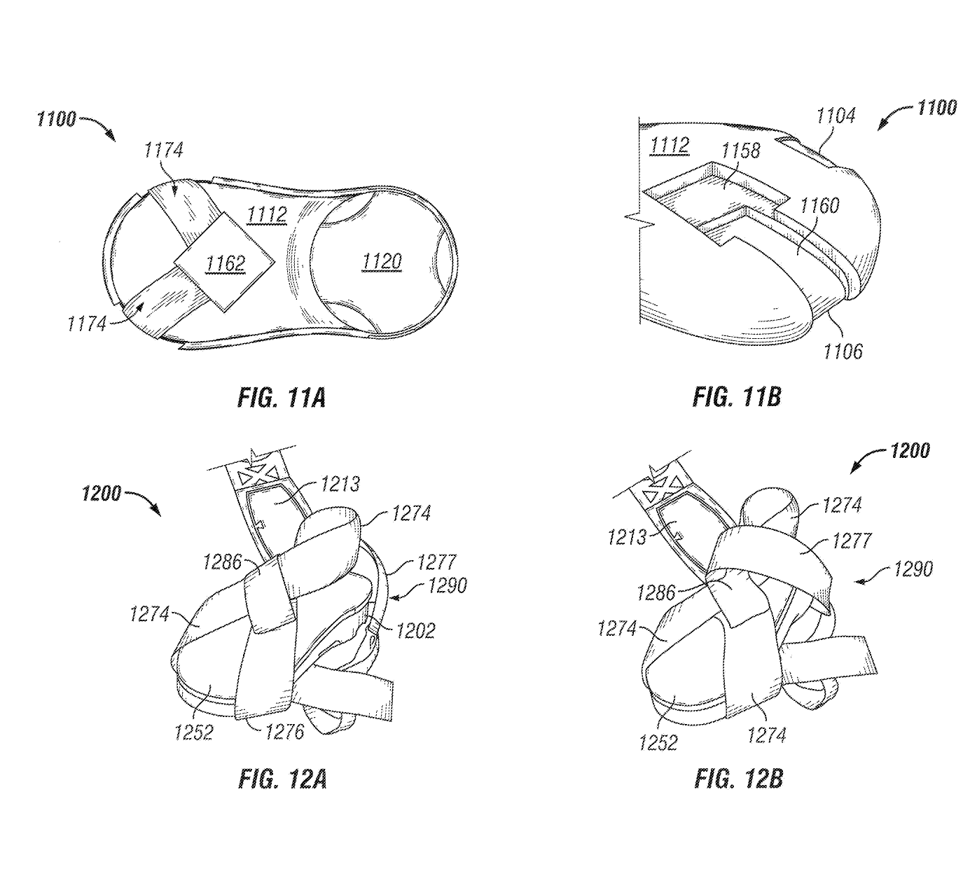

The slot 358 can be used to receive a buckle (described below with respect to FIG. 11A). In such a case, the buckle can be mechanically coupled to the slot 358 using one or more of a number of methods, including but not limited to fastening devices (e.g., Velcro, screws), slotted fittings, epoxy, and overmolding. As shown in FIGS. 3C and 3E, complementary to the slot 358 is a notch 304 disposed on a side of the foot plate 300 along the path of the buckle when the buckle is positioned within the slot 358. The notch 304 is disposed along the path of the slot 358 along the outer edge of the foot plate 300. In certain exemplary embodiments, the notch 304 complements the slot 358 in guiding the securing member.

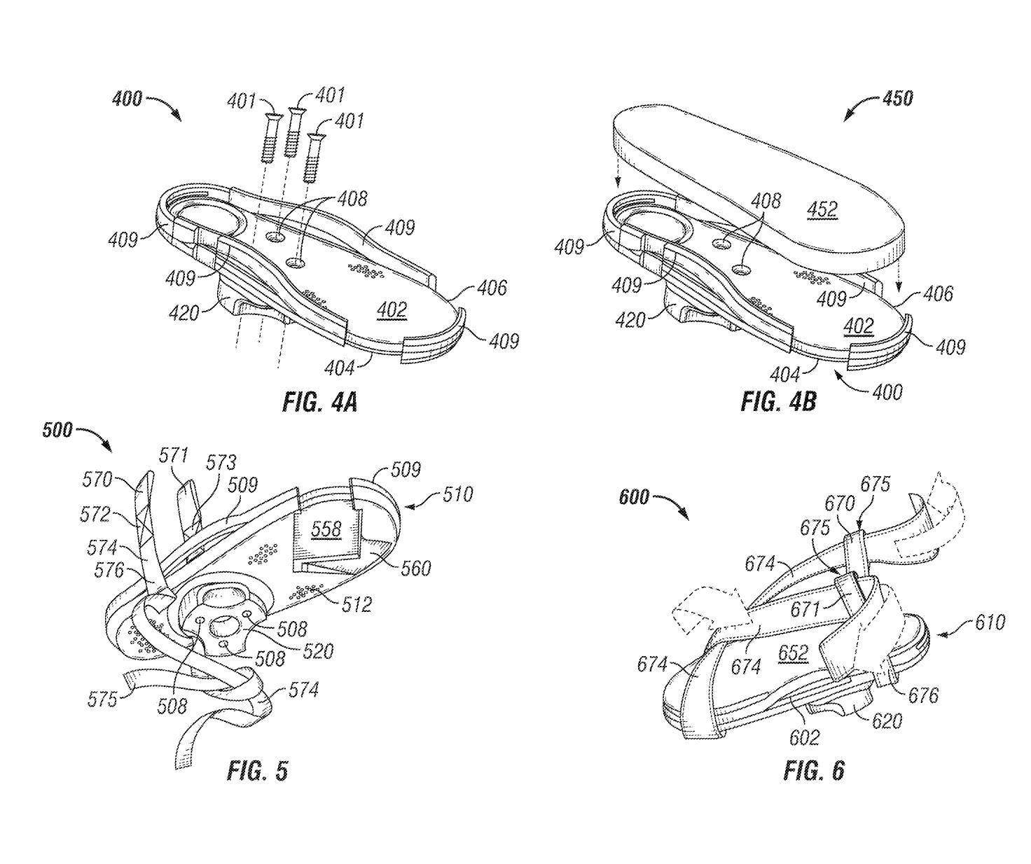

The top side 302, the bottom side 312, and/or the sides of the foot plate 300 can include one or more other features. Specifically, the top side 302, the bottom side 312, and/or the sides can have a receiving feature (not shown in FIGS. 3A-E) for one or more components of the foot restraint 150. For example, the top side 302 can include a Velcro element that mates to a corresponding Velcro element of the insert 152, a side support 154, and/or some other component. As another example, the top side 302 can include a slotted feature that receives a corresponding protruding member of the insert 152, a side support 154, and/or some other component. Additional exemplary features of the foot plate 300 are shown in FIGS. 4A and 4B below

FIGS. 4A and 4B show various views of another exemplary foot plate 400 for a brace used to correct clubfoot in accordance with certain exemplary embodiments. Specifically, FIG. 4A shows a top perspective view of the foot plate 400 without the insert 452, and FIG. 4B shows a top perspective view of the foot plate 450 with the insert 452. The foot plate 400 of FIGS. 4A and 4B is substantially the same as the foot plate described above with respect to FIGS. 3A-E.

The footplate 400 includes additional features not found in the foot plate 300 of FIGS. 3A-3E. Specifically, one or more insert guides 409 can be mounted along all or a portion of the perimeter of the foot plate 400 and extend upward from the top side 402 of the foot plate 400. The insert guides 409 are used to secure the insert 452 against the top side 402 of the foot plate 400. The insert guides 409 and the foot plate 400 can be a molded single body construction. Alternatively, the insert guides 409 can be separate components that are mechanically coupled to the foot plate 400. The mechanical coupling can be fixed, removable, some other suitable type of coupling, or any combination thereof. The insert guides 409 can be mechanically coupled to the foot plate 400 using one or more of a number of methods, including but not limited to fastening devices (e.g., Velcro, screws), slotted fittings, epoxy, and overmolding.

The insert guides 409 can be made of one or more of a number of suitable materials, including but not limited to plastic, rubber, and metal. The material of the insert guides 409 can be the same or different than the materials of the foot plate 400. The height, width, and other dimensions of the insert guides 409 can vary, depending on one or more of a a number of factors. Such factors include, but are not limited to, the shape of the insert 452, the height of the insert 452, the shape of the top side of the foot plate 400, the size and/or shape of the foot, and the material of the insert 452. The insert guides 409 can be rigid and/or flexible. The insert guides 409 can include one or more features (e.g., Velcro, ridges, slots) used to secure the insert 452.

Multiple insert guides 409 can be disposed along the perimeter of the foot plate 400 in non-continuous segments. For example, as shown in FIGS. 4A and 4B, the insert guides 409 can have gaps between them that create notches 404 and 406, which can coincide with slot and the channel, respectively, disposed on the bottom side of the foot plate 400.

FIG. 4A also shows the fastening devices 401, in the form of bolts, prior to being inserted through the apertures 408 in the foot plate 400 and the mounting feature 420. In certain exemplary embodiments, the insert 452 is removably coupled to the top side 402 of the foot plate 400. When the insert 452 is removed from the top side 402 of the foot plate 400, the fastening devices 401 are exposed and accessible.

FIG. 5 shows an exemplary foot restraint 500 in accordance with certain exemplary embodiments. The foot plate 510 of FIG. 5 is substantially the same as the foot plate 400 described above with respect to FIGS. 4A and 4B. In addition, the foot restraint 500 of FIG. 5 includes a number of securing members 574, 575.

Each securing member 574, 575 is used to contact and secure at least a portion of the foot. The securing member 574, 575 is made of one or more flexible materials including but not limited to nylon, cotton, and polyester. For example, the securing members 574, 575 can be nylon straps. In certain exemplary embodiments, there is a single securing member for each foot restraint. In other exemplary embodiments, there are multiple securing members for each foot restraint.

At least part of the securing member 574, 575 can be coupled to the foot plate 510 and/or the insert guides 509. The coupling between the securing member 574, 575 and the foot plate 510 and/or the insert guides 509 can be fixed, removable, slidable, some other suitable type of coupling, or any combination thereof. The securing member 574, 575 can be coupled to the foot plate 510 and/or the insert guides 509 using one or more of a number of methods, including but not limited to fastening devices (e.g., Velcro, screws), slotted fittings, epoxy, stitching, and overmolding.

Each securing member 574, 575 can have one or more features along various portions of the length of the securing member 574, 575. For example, as shown in FIG. 5, the top end of the securing members 574, 575 is folded over on themselves to form a loop 570, 571. The loop 570, 571 is large enough to receive a different portion of the securing member 574, 575 so that the securing member 574, 575 slidably couples with the loop 570, 571. The loop 570, 571 is formed by coupling the top end of the securing member 574, 575 to another portion of the securing member 574, 575. In FIG. 5, the top end of the securing member 574 is sewn to the securing member 574 at location 572. In addition, or in the alternative, the top end of the securing member 574, 575 can be coupled to another portion of the securing member 574, 575 in one or more other ways, including but not limited to using a fastening device (e.g., a clip, a buckle, a rivet, a snap) and using epoxy.

Alternatively, or in addition, a portion of the securing member 574, 575 can be coupled to at least one other component of the foot restraint. Examples of such other component can include, but are not limited to, the foot plate, a channel, a buckle, a loop, and a different portion of the securing member 574. Such examples are described below. When such other component to which the securing member 574 is coupled is the foot plate 510, as shown in FIG. 5, the coupling can occur at one or more different locations on the foot plate 510. For example, the securing member 574 can be coupled to the side of the foot plate 510, such as at location 576 shown in FIG. 5. As another example, the securing member 574 can be coupled to the side of the foot plate at a location along the bottom side 512 of the foot plate 510.

The securing member 574 also has a length that extends below the coupling points 572 and/or 576, allowing the securing member 574 to secure a foot in the foot restraint 500 by wrapping around the foot and the foot plate 510. In certain exemplary embodiments, the securing members 574 and 575 are the same securing member (i.e., a single piece) rather than separate pieces. In addition, or in the alternative, the securing members 574, 575 forming the loop 570, 571 are the same securing member (i.e., a single piece) rather than separate pieces.

Further, in addition or in the alternative, the securing member 574 forming the loop 570 can be a separate piece from the securing member 574 that secures the foot to the foot restraint 500. In such a case, for example, the separate securing members can be coupled at location 576. Similarly, the securing member 575 forming the loop 571 can be the same or a different piece from the securing member 575 that secures the foot to the foot restraint 500. If separate securing members are coupled to each other, the coupling can occur in one or more of a number of ways, including but not limited to stitching, epoxy, Velcro, snap fittings, and interlocking clips. Such a coupling can be fixed, detachable, some other type of coupling, or any suitable combination thereof.

FIG. 6 shows a foot restraint 600 where one securing member 675 is fixedly coupled on both sides of the foot plate 610, where one coupling is at location 676 and the other coupling is hidden from view on the opposite side of the foot plate 610. Such couplings to the side of the foot plate 610 are substantially an equal distance from the center of the length of the securing member 675. In such a case, the bottom side (hidden from view) of the foot plate 610 may have a channel to guide the securing member 675 across the width of the foot plate 610. Similar to the loop 570, 571 shown in FIG. 5 above, each end of the securing member 675 in FIG. 6 is coupled to another portion of the securing member 675 at a location (hidden from view) below the loop 670, 671 formed by such coupling.

Both loops 670, 671 of the securing member 675 in FIG. 6 are slidably coupled to a different securing member 674. A portion of the securing member 674 is directed through the channel and/or slot (not shown) in the bottom side of the foot plate 610. Further, other portions of the securing member 674 traverse the loops 670, 671 in the securing member 675 to secure the foot to the foot restraint 600.

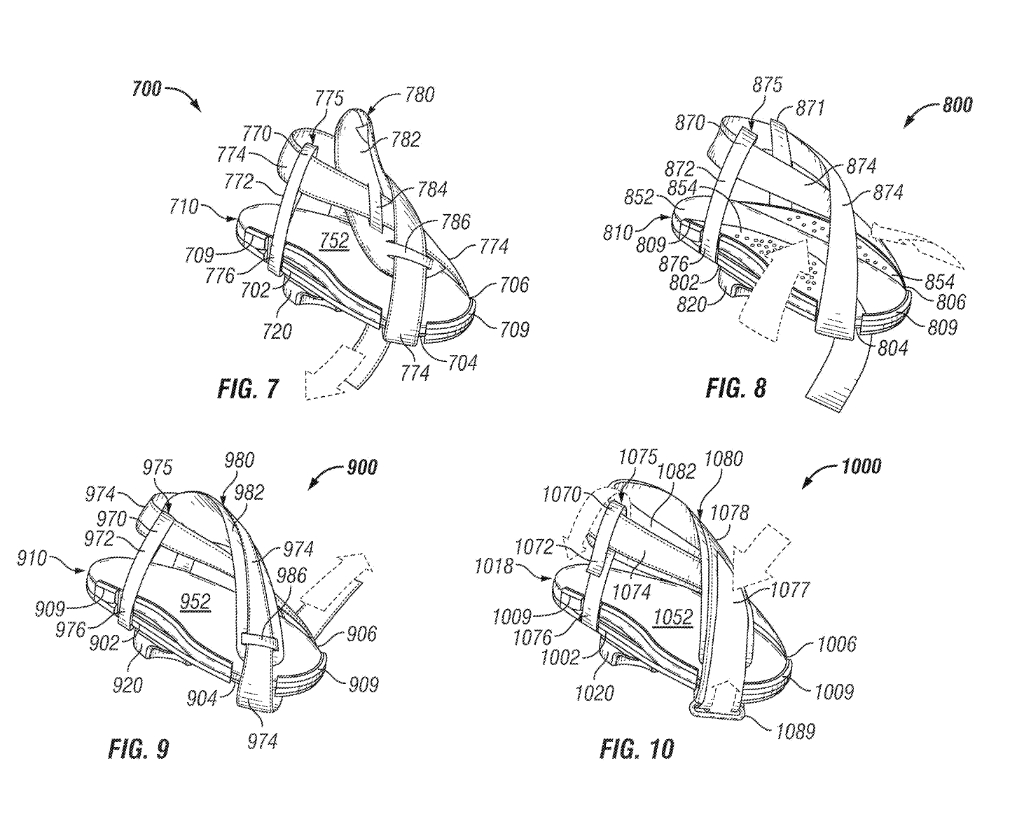

FIG. 7 shows an exemplary foot restraint 700 for a brace used to correct clubfoot in accordance with certain exemplary embodiments. The foot restraint 700 of FIG. 7 is substantially the same as the foot restraint 600 described above with respect to FIG. 6. In addition, the foot restraint 700 of FIG. 7 includes a sorting member 780. Specifically, the sorting member 782 is slidably coupled to the securing member 774. The sorting member 782 can be a separate piece with at least one slot 784, 786 through which one or more securing members (e.g., securing member 774) traverse to create a slidable coupling. In this example, the sorting member 782 is shaped like, and positioned similar to, the tongue of a traditional lace-up shoe.

The sorting member 780 can also have a front side 782 and a back side (not shown). The back side of the sorting member 782, which contacts the foot when the foot is placed within the foot restraint 700, can be padded. The sorting member 780 can be made of one or more of a number of materials, including but not limited to leather, nylon, rubber, cotton, polyester, and foam. While FIG. 7 shows that the sorting member 780 has two slots 784, 786, the sorting member 780 can have a single slot or more than two slots.

Alternatively, or in addition, the sorting member 780 can be coupled to one or more other members of the foot restraint 700, including but not limited to the supporting member 775, a different portion of the securing member 774, the foot plate 710, and the insert guides 709. In addition to, or in the alternative to, being slidably coupled, the sorting member 780 can be coupled to a component of the foot restraint 700 detachably, fixedly, in some other manner, or any combination thereof.

FIG. 8 shows another exemplary foot restraint 800 for a brace used to correct clubfoot in accordance with certain exemplary embodiments. The foot restraint 800 of FIG. 8 is substantially the same as the foot restraint 700 of FIG. 7 without the sorting member 782 and with the side supports 154 described above with respect to FIGS. 1A and 1B.

FIG. 9 shows yet another exemplary foot restraint 900 for a brace used to correct clubfoot in accordance with certain exemplary embodiments. The foot restraint 900 of FIG. 9 is substantially the same as the foot restraint 700 of FIG. 7 except that the sorting member 980 is coupled to a portion of the length of the supporting member 972. In this case, the sorting member 980 has a width that is wider than the width of the supporting member 972 where the sorting member 980 is coupled to the supporting member 972. The sorting member 980 can be a separate piece with at least one slot 986 through which one or more securing members (e.g., securing member 974) traverse to create a slidable coupling.

The sorting member 980 can also include a front side 982 and a back side (not shown). The back side of the sorting member 982, which contacts the foot when the foot is placed within the foot restraint 900, can be padded. The sorting member 980 can be made of one or more of a number of materials, including but not limited to leather, nylon, rubber, cotton, polyester, and foam. While FIG. 9 shows that the sorting member 980 has one slot 986, the sorting member 980 can have two or more slots.

Alternatively, or in addition, the sorting member 980 can be coupled to one or more other members of the foot restraint 900, including but not limited to the supporting member 975, a different portion of the securing member 974, the foot plate 910, and the insert guides 909. In addition to, or in the alternative to, being slidably coupled, the sorting member 980 can be coupled to a component of the foot restraint 900 detachably, fixedly, in some other manner, or any combination thereof.

FIG. 10 shows another exemplary foot restraint 1000 for a brace used to correct clubfoot in accordance with certain exemplary embodiments. The foot restraint 1000 of FIG. 10 is substantially the same as the foot restraint 900 of FIG. 9, except that the sorting member 1080 couples to the back side of the supporting member 1072 for a longer segment than shown in FIG. 9.

In addition, the end of the securing member 1075 in FIG. 10 couples to another portion of the securing member 1075 at location 1072 using Velcro to form the loop 1070. In this way, the size of the loop 1070 through which the securing member 1074 slidably couples can be adjustable. In addition, by having a removable coupling where the portions of the securing member 1075 form the loop 1070, adjustments to one or more portions of the foot restraint 1000 can be made more easily. As shown in FIG. 10, the loop 1070 can be disposed between the back side of the securing member 1075 and the front side of the sorting member 1080. Alternatively, the loop 1070 can be disposed behind the back side of the sorting member 1080.

In addition, the distal end of the securing member 1075 passes through a loop 1089 and is secured to another portion of the securing member 1075 using Velcro. The loop 1089 can be coupled to a portion (e.g., the bottom side, the side) of the foot plate 1010. The loop 1089 can also be coupled to one or more other components of the foot assembly 1000, including but not limited to one or more insert guides 1009. The loop has dimensions (e.g., width, height) sufficient to allow the securing member 1075, as well as any sorting member 1080 coupled to the securing member 1075, to pass therethrough. The loop 1089 can be coupled to the foot plate in one or more of a number of ways, including but not limited to fixedly, detachably, slidably, and hingedly. The loop 1089 can be of one or more of a number of suitable materials, including but not limited to plastic, nylon, and metal.

FIGS. 11A and 11B show the bottom side 1112 of an exemplary foot plate 1100 in accordance with certain exemplary embodiments. Specifically, FIG. 11A shows a buckle 1162 disposed within the slot 1158, shown in FIG. 11B. The buckle 1162 can be coupled to the slot 1158 in one or more of a number of ways, including but not limited to fixedly, detachably, slidably, and hingedly. The buckle 1162 can be of one or more of a number of suitable materials, including but not limited to plastic, nylon, rubber, and metal.

In certain exemplary embodiments, the buckle 1162 fixedly and/or detachably couples to at least one securing member (e.g., securing member 1174). For example, the buckle 1162 can be fixedly coupled to one end of the securing member 1174, where the securing member 1174 is directed into the channel 1160 on the bottom side 1112 of the foot plate 1110 and past the notch 1106 in the side of the foot plate 1110. The opposite end of the securing member 1174 can be directed through notch 1104 and slidably coupled to a different portion of the buckle 1162. Such different portion of the buckle 1162 can have a number of different coupling configurations, including but not limited to a locking hinge, a latch (which would be inserted into one of a number of holes in the securing member 1174), a clamp, and Velcro. In any case, the buckle 1162 can intermittently couple and decouple to the securing member 1174 to allow the securing member 1174 to have an appropriate length to properly secure a foot in the foot restraint.

FIGS. 12A and 12B show a top perspective view of a portion of an exemplary brace 1200 in accordance with certain exemplary embodiments. In this example, there are two securing members: securing member 1274 and securing member 1277. Securing member 1277 is coupled to either side and/or the bottom side of the foot plate 1202. The mechanical coupling between the securing member 1277 and the foot plate 1202 allows the securing member 1277 to rotate between the toe and the heel of the foot plate 1202. The securing member 1277 in this case forms a continuous loop, but can also be made up of two or more pieces coupled in some suitable manner. Further, the coupling of one or both ends of the securing member 1277 to the foot plate 1202 can be adjustable, to allow the loop formed by the securing member 1277 to increase and/or decrease.

The securing member 1274 is coupled on both ends to a buckle (hidden from view) disposed on the bottom side of the foot plate 1202 in a manner similar to that described above with respect to the buckle 1162 of FIGS. 11A and 11B. The coupling of one or both ends of the securing member 1274 to the buckle can be adjustable, allowing the amount of the securing member 1277 used to secure a foot to the foot restraint 1290 to increase and/or decrease.

In addition, supporting member 1286 is fixedly coupled to a portion of the securing member 1274. In this example, the supporting member 1286 is a cloth (e.g., nylon, polyester) segment that is sewn at each end to the securing member 1274. In such a case, a different portion of the securing member 1274 can slide between the bottom side of the supporting member 1286 and the top side of a different portion of the securing member 1274. This slidable coupling allows the securing member 1274 to be properly aligned in securing the foot in the foot restraint 1290.

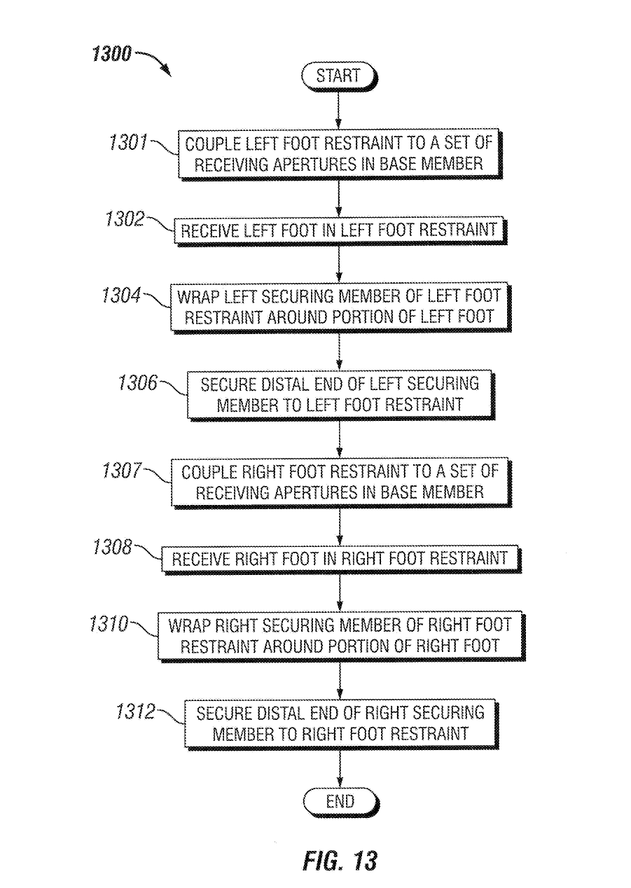

FIG. 13 is a flowchart of a method 1300 for correcting clubfoot in accordance with certain exemplary embodiments. While the various steps in this flowchart are presented and described sequentially, one of ordinary skill will appreciate that some or all of the steps may be executed in different orders, may be combined or omitted, and some or all of the steps may be executed in parallel. For example, while Steps 1301-1306 are shown in FIG. 13 and described below as occurring in series prior to Steps 1307-1312, Steps 1307-1312 may be performed prior to Steps 1301-1306.

Further, in certain of the exemplary embodiments, certain of the steps described below may be omitted, repeated, and/or performed in a different order. In addition, a person of ordinary skill in the art will appreciate that additional steps, not shown in FIG. 13, may be included in performing this method. Accordingly, the specific arrangement of steps should not be construed as limiting the scope.

Now referring to FIGS. 1-13, the exemplary method 1300 begins at the START step and proceeds to Step 1301, where a left foot restraint is coupled to one of at least two sets of receiving apertures 115, 116 in a base member 110 to create an outward angle of the foot restraint 150 relative to a perpendicular angle. The perpendicular angle may also be referred to as a normal angle and is the angle perpendicular to the axis along the length of the base member 110. The outward angles can be discrete angles that are predetermined based on the orientation of the sets of receiving apertures 115, 116. For example, one outward angle corresponding to one set of receiving apertures 116 can be 30°, and another outward angle corresponding to another set of receiving apertures 115 can be 60°.

In step 1302, a left foot is received in a left foot restraint 500 of the brace 100. In certain exemplary embodiments, the left foot is experiencing a clubfoot condition, and the brace 100 is used to correct the clubfoot condition. In step 1304, a first securing member 574 of the left foot restraint 500 is wrapped around a portion of the left foot. In certain exemplary embodiments, the left securing member 574 also wraps around one or more portions of the foot plate 510. Further, one or more other securing members (e.g., securing member 575) can be wrapped around and/or contact one or more portions of the foot. In addition, or in the alternative, a sorting member 780 can be used to align one or more of the supporting members. At least one end of the left securing member 574 is coupled (e.g., fixedly, slidably, removably) to a portion of the foot restraint 500. For example, one end of the left securing member 1174 can be fixedly coupled to a buckle 1162 that is slidably coupled to a slot 1158 in the bottom side 1112 of the foot plate 1100.

In wrapping the first securing member 574 of the left foot restraint 500 around the left foot, the first securing member 574 can overlap with itself and/or with one or more other components of the left foot restraint 500. For example, the securing member 1274 can overlap itself using a sorting member 1286.

In step 1306, a distal end of the left securing member 574 is secured to the left foot restraint 500. In certain exemplary embodiments, the distal end of the left securing member 574 is secured to the left foot restraint 500 after the left securing member 574 is wrapped around a portion of the left foot. The distal end of the left securing member 574 can be secured in a number of ways. For example, if a buckle 1162 is disposed on a bottom side 1112 of the foot plate 1100, the distal end of the securing member 1174 can be passed through the buckle 1162 coupled to the slot 1158 in the bottom side 1112 of the foot plate 1100. The distal end of the securing member 1174 is then pulled through the buckle 1162 until the securing member 1174 snugly secures the left foot in the foot restraint 500. Finally, a latch of the buckle 1162 is secured.

As another example, if a loop 1089 is coupled to a portion of the foot plate 1010, the distal end of the securing member 1074 can be passed through the loop 1089. Subsequently, the distal end of the securing member 1074 is then pulled through the loop 1089 until the securing member 1074 snugly secures the left foot in the foot restraint 1000. Finally, a Velcro surface disposed on the distal end of the securing member 1074 can be contacted to a corresponding Velcro surface disposed on a different portion of the securing member 1074 to secure the securing member 1074 in place.

In step 1307, a right foot restraint is coupled to one of at least two sets of receiving apertures 115, 116 in a base member 110 to create an outward angle of the foot restraint 150 relative to the perpendicular angle. In step 1308, a right foot is received in a right foot restraint 500 of the brace 100. In step 1310, a second securing member 574 of the right foot restraint 500 is wrapped around a portion of the right foot. In step 1312, a distal end of the right securing member 574 is secured to the right foot restraint 500. These steps 1307-1312 for the right foot restraint are substantially similar to steps 1301-1306, respectively, for the left foot restraint. The process then continues to the END step.

Using exemplary embodiments described herein, the brace for correcting a clubfoot condition uses inexpensive and lightweight materials, making the brace lighter, less expensive, easier to use, and easier to wear. The exemplary brace is also more comfortable for the infant or child patient, making treatment easier to administer. Further, because of the simplistic design, the brace is easy to administer and requires little or no intervention of a medical professional. Because the brace is less expensive but still extremely effective in treating clubfoot, more children afflicted with clubfoot can be treated and cured, despite a lack of access to insurance and/or financial resources. Consequently, fewer people will suffer from the effects of clubfoot, reducing long-term medical costs and other costs associated with the inability to walk.

Although embodiments described herein are made with reference to exemplary embodiments, it should be appreciated by those skilled in the art that various modifications are well within the scope and spirit of this disclosure. Those skilled in the art will appreciate that the exemplary embodiments described herein are not limited to any specifically discussed application and that the embodiments described herein are illustrative and not restrictive. From the description of the exemplary embodiments, equivalents of the elements shown therein will suggest themselves to those skilled in the art, and ways of constructing other embodiments using the present disclosure will suggest themselves to practitioners of the art. Therefore, the scope of the exemplary embodiments is not limited herein.

We claim:

1. A brace to correct a clubfoot condition, the brace comprising:
a pair of foot restraints, wherein each foot restraint comprises:
a foot plate comprising a mounting feature disposed on a bottom side of the foot plate and a plurality of apertures that traverse the mounting feature and the foot plate;
a first securing member with a first portion mechanically coupled to the foot plate; and
an insert removably coupled to a top side of the foot plate; and
a base member mechanically coupled to each of the mounting features at each end of the base member, wherein each end of the base member comprises a coupling feature, wherein each coupling feature comprises at least two sets of receiving apertures.

2. The brace of claim 1, further comprising a plurality of fastening devices that traverse the plurality of apertures in the mounting feature and the foot plate and one of the at least two sets of receiving apertures in the coupling features of the base member.

3. The brace of claim 2, wherein the pair of foot restraints form an outward angle with the base member when the pair of foot restraints are mechanically coupled with the coupling features of the base member.

4. The brace of claim 3, wherein the outward angle is one selected from a group consisting of 30° and 60°, wherein the outward angle of 30° corresponds to the first set of receiving apertures in the base member, and wherein the outward angle of 60° corresponds to a second set of receiving apertures in the base member.

5. The brace of claim 2, wherein the plurality of fastening devices are accessible when the insert is removed.

6. The brace of claim 1, wherein the base member further comprises a center portion that creates a dorciflexion angle when the pair of foot restraints are mechanically coupled with the coupling features of the base member.

7. The brace of claim 1, wherein the bottom side of the foot plate comprises a channel for guiding the first securing member.

8. The brace of claim 7, further comprising a buckle positioned within a slot disposed on the bottom side of the foot plate, wherein the buckle secures a second portion of the first securing member, and wherein the slot is adjacent to the channel.

9. The brace of claim 1, further comprising a loop fixedly coupled to the bottom side of the foot plate, wherein the first securing member passes through the loop.

10. The brace of claim 1, further comprising a second securing member having a first end fixedly coupled to the foot plate and a second end, wherein the first securing member slidably couples to the second end of the second securing member at a second portion.

11. The brace of claim 1, wherein the base member has an adjustable length.

12. The brace of claim 1, wherein the base member comprises a honey comb shape.

13. The brace of claim 1, wherein the base member comprises a crisscross rib structure.

14. The brace of claim 1, wherein the first securing member comprises an attaching feature at a distal end, wherein the attaching feature mechanically couples to a second portion of the first securing member.

15. The brace of claim 1, further comprising a sorting member slidably coupled to at least a second portion of the first securing member.

16. A method to correct a clubfoot condition using a brace, the method comprising:
receiving a left foot into a left foot restraint;
wrapping a first securing member of the left foot restraint around a first portion of the left foot;
securing, after wrapping the left securing member around the first portion of the left foot, a first distal end of the left securing member to the left foot restraint;
receiving a right foot into a right foot restraint;
wrapping a second securing member of the right foot restraint around a second portion of the right foot; and
securing, after wrapping the second securing member around the second portion of the right foot, a second distal end of the second securing member to the right foot restraint.

17. The method of claim 16, further comprising coupling the left foot restraint to one of at least two sets of receiving apertures in a base member to create an outward angle of the foot restraint relative to a perpendicular angle, wherein the outward angle comprises at least one selected from a group consisting of 30° and 60°.

18. The method of claim 16, wherein wrapping the first securing member of the left foot restraint around the first portion of the left foot comprises overlapping at least a first portion of the left foot restraint with at least a second portion of the left foot restraint.

19. The method of claim 16, wherein securing the first distal end of the left securing member to the left foot restraint comprises:
passing the first distal end of the left securing member through a buckle coupled to a bottom portion of the left foot restraint;
pulling the first distal end of the left securing member until the left securing member snugly secures the left foot; and
securing a latch on the buckle.

20. The method of claim 16, wherein securing the first distal end of the left securing member to the left foot restraint comprises:
passing the first distal end of the left securing member through a loop coupled to a bottom portion of the left foot restraint;
pulling the first distal end of the left securing member until the left securing member snugly secures the left foot; and
contacting a Velcro surface disposed on the distal end of the left securing member to a complementary Velcro surface disposed on a first portion of the left securing member.
